(19) United States
(12) Patent Application Publication
(10) Pub. No.: US 2018/0111906 A1
(43) Pub. Date: Apr. 26, 2018

(54) IMPROVED PROCESS FOR PREPARING A STATIN PRECURSOR

(71) Applicant: DSM Sinochem Pharmaceuticals Netherlands B.V., Delft (NL)

(72) Inventors: Ben De Lange, Echt (NL); Karin Henderika Maria Bessembinder, Echt (NL); Dennis Heemskerk, Echt (NL)

(21) Appl. No.: 15/561,977

(22) PCT Filed: Mar. 24, 2016
(86) PCT No.: PCT/EP2016/056632

§ 371 Date: Sep. 26, 2017

(30) Foreign Application Priority Data
Mar. 26, 2015 (EP) 15160957.5

(51) Int. Cl. C07D 239/69; C07D 239/34; C07D 405/06

(52) U.S. Cl. CPC C07D 239/69; C07D 405/06; C07D 239/34

(57) ABSTRACT

The present invention relates to a process comprising reacting p-fluorobenzaldehyde, 4-methyl-3-oxopentanenitrile and urea in a first reaction mixture comprising a first organic solvent, thereby obtaining an intermediate mixture comprising an oxo-pyrimidine-carbonitrile; and oxidizing the oxo-pyrimidine-carbonitrile by contacting the intermediate mixture or organic extract with an organic hydroperoxide, thereby obtaining a hydroxyl-pyrimidine carbonitrile.

### FIELD OF THE INVENTION

The invention is directed to a process for preparing a statin precursor. In particular, the invention is directed to a process for preparing 4-(4-fluorophenyl)-2-hydroxy-6-isopropylpyrimidine-5-carbonitrile.

### BACKGROUND OF THE INVENTION

Rosuvastatin, in particular rosuvastatin calcium, is a well-known HMG-CoA reductase inhibitor which is used for the treatment of hypercholesterolemia and to prevent cardiovascular disease. The compound according to formula (I) is a well-known precursor for preparing rosuvastatin.

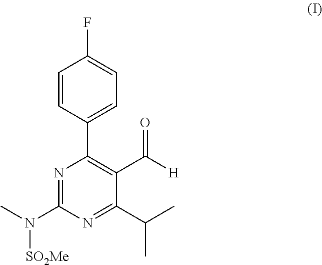 [SMILES: CC(C)c1nc(N(C)S(C)(=O)=O)nc(-c2ccc(F)cc2)c1C=O]

Different processes are known to prepare the compound of formula (I). One such process is described in WO 2008/151510, wherein the compound of formula (I) is prepared from p-fluorobenzaldehyde, 4-methyl-3-oxopentanenitrile and urea. This process is represented in the reaction scheme A below.

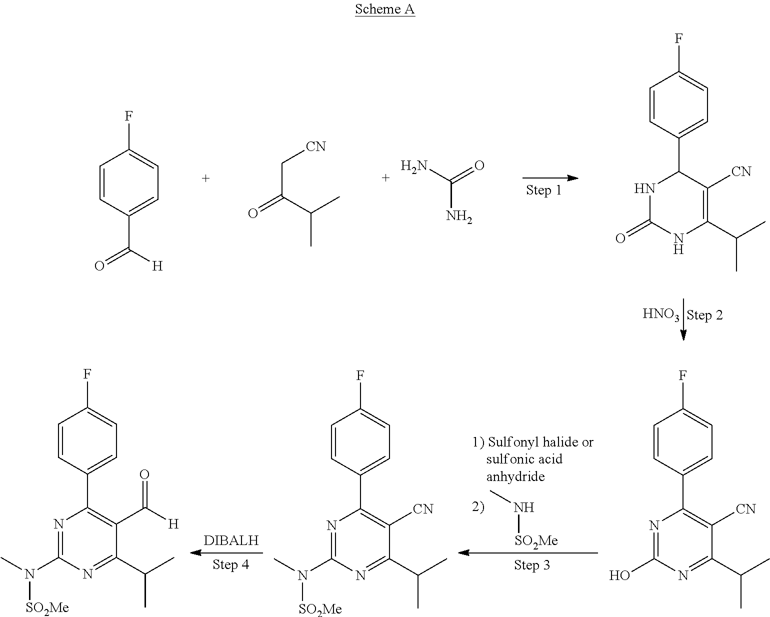

In the first step of WO 2008/151510, p-fluorobenzaldehyde, 4-methyl-3-oxopentanenitrile and urea are reacted to obtain an oxo-pyrimidine-carbonitrile according to formula (II).

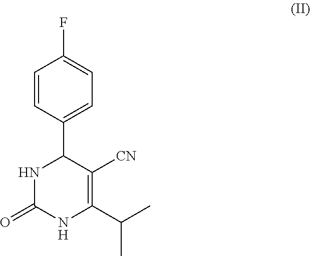 [SMILES: CC(C)C1=C(C#N)C(c2ccc(F)cc2)NC(=O)N1]

This compound is isolated from the reaction mixture and subsequently oxidized with HNO₃ to form the hydroxy-pyrimidine-carbonitrile according to formula (III).

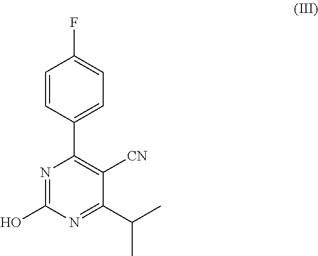 [SMILES: CC(C)c1nc(O)nc(-c2ccc(F)cc2)c1C#N]

A disadvantage of step 1 of the process of WO 2008/151510 is that the compound of formula (II) is recovered with a relatively low yield (as illustrated by Example 1 below).

### DETAILED DESCRIPTION OF THE INVENTION

An object of the invention is to increase the yield obtained in step 1 outlined above. The inventors found that the low yield of step 1 of reaction scheme A was caused not so much by the efficiency of the reaction, but rather due to the difficulty in isolating the oxo-pyrimidine-carbonitrile crystals from the reaction mixture. However, it proved difficult to improve the isolation. In particular, the separation of the oxo-pyrimidine-carbonitrile from unreacted urea could not be improved in a satisfying way. The inventors conducted experiments that showed that the product loss during isolation was about 20%, which makes it impossible to obtain a good overall yield.

The inventors found that by conducting step 2 of reaction scheme A using a specific oxidizing agent, steps 1 and 2 could be integrated with each other, such that isolation of oxo-pyrimidine-carbonitrile crystals was no longer necessary. This should provide for an effective increase in the yield of step 1.

Accordingly, the invention is directed to a process comprising the steps of:
reacting p-fluorobenzaldehyde, 4-methyl-3-oxopentanenitrile and urea in a reaction mixture comprising a first organic solvent, thereby obtaining an intermediate mixture comprising the compound of formula (II) dissolved in the first organic solvent; and
oxidizing the compound of formula (II) with an organic hydroperoxide, thereby obtaining the compound of formula (III),

wherein the compound of formula (II) is kept in dissolved form between its formation and oxidation.

The inventors expect that it is possible to obtain a considerably higher yield of the compound of formula (III) with the process of the invention compared to the prior art process known from WO 2008/151510, based on the results of Examples 3 and 4. The process of the invention makes it possible to keep the compound of formula (II) in dissolved form throughout the process, such that it does not have to be isolated in solid form. Thus, a higher yield can be obtained.

In order to keep the compound of formula (II) in dissolved form between its formation and oxidation, the compound may be kept in solution in the first organic solvent until oxidation. The oxidation step will thus be conducted in the same solvent as the organic solvent used in the first reaction step, without having to isolate the compound of formula (II) in solid form. In this case, the process of the invention comprises the steps of:
(a) reacting p-fluorobenzaldehyde, 4-methyl-3-oxopentanenitrile and urea in a reaction mixture comprising a first organic solvent, thereby obtaining an intermediate mixture comprising the compound of formula (II) dissolved in the first organic solvent; and
(b) oxidizing the compound of formula (II) by contacting the intermediate mixture with an organic hydroperoxide, thereby obtaining the compound of formula (III).

Alternatively, the compound of formula (II) may be transferred from the first organic solvent to a second organic solvent, while keeping the compound in dissolved form, e.g. by extraction. Oxidation may then be conducted in the second organic solvent. In case the compound of formula (II) is extracted to a second organic solvent, the process of the invention comprises the steps of:
(a1) reacting p-fluorobenzaldehyde, 4-methyl-3-oxopentanenitrile and urea in a reaction mixture comprising a first organic solvent, thereby obtaining an intermediate mixture comprising the compound of formula (II) dissolved in the first organic solvent; and
(a2) extracting the compound of formula (II) from the intermediate mixture to a second organic solvent, thereby forming an organic extract comprising the compound of formula (II) dissolved in the second organic solvent; and
(b) oxidizing the compound of formula (II) by contacting the organic extract with an organic hydroperoxide, thereby obtaining the compound of formula (III).

According to the process of the invention, the compound of formula (II) is kept in dissolved form between its formation and oxidation. This means that when the compound of formula (II) is formed in the first reaction step (from p-fluorobenzaldehyde, 4-methyl-3-oxopentanenitrile and urea), the compound is kept in solution until it is oxidized by the organic hydroperoxide in the oxidation step. Thus, the compound of formula (II) is in dissolved form during the process. In particular, the compound of formula (II) is not isolated in solid form at any point in the process (e.g. not from the reaction mixture, nor from the extract). Typically, the compound of formula (II) is either in dissolved form, because it is dissolved in the first organic solvent or because it is dissolved in in the second organic solvent.

An important part of the invention lies in the selection of the oxidizing agent. The oxidizing agent should be able to efficiently convert the oxo-pyrimidine-carbonitrile into the hydroxyl-pyrimidine carbonitrile with a high yield, even in the presence of organic solvent. Since the oxo-pyrimidine-carbonitrile is contacted with the oxidizing agent while being dissolved in an organic solvent, the oxidation will necessarily be conducted in the presence of organic solvent. Accordingly, if the oxidizing agent is not suitable for oxidizing the oxo-pyrimidine-carbonitrile efficiently and/or safely in the presence of the organic solvent, the oxidizing agent cannot be used. For example, the oxidizing agent HNO₃ (which is used in step 2 in WO 2008/151510) is not suitable for use in the process of the invention for this reason. HNO₃ reacts violently and hypergolic with most such organic solvents, such that this oxidizing agent cannot be used to oxidize oxo-pyrimidine-carbonitrile when present in an organic solvent.

The inventors found that using an organic hydroperoxide as the oxidizing agent resulted in a very good oxidation yield, even when conducted in the presence of an organic solvent. Accordingly, the use of such an oxidizing agent makes it possible to use oxo-pyrimidine-carbonitrile in dissolved form as a reactant, thereby eliminating the need for the yield reducing isolation of oxo-pyrimidine-carbonitrile.

The organic hydroperoxide may have the formula R—O—O—H, wherein R is an organic group. The organic group may be an aliphatic or an aromatic group, such as an alkyl or aryl group. The organic group may have from 1 to 20, preferably from 1 to 12 carbon atoms. The organic group may be a substituted or unsubstituted group. The organic group may be an aromatic hydrocarbon (preferably cumene), alkane (preferably butyl) or cycloalkane. In case the organic group is substituted, the organic group may be substituted with one or more substituting groups, which are preferably selected from C1-C4 alkyl (e.g. methyl or ethyl) or halide (e.g. Cl, Br, F, I). The organic group may further comprise one or more oxygen atoms, for example as part of a heterocyclic group.

Examples of suitable aliphatic hydroperoxide are alkyl hydroperoxides. Examples of suitable aromatic hydroperoxides are cumene hydroperoxide and isopropylcumene hydroperoxide. Preferably, alkyl hydroperoxides are used, wherein the alkyl group may have from 1 to 12 carbon atoms, preferably 1-6 carbon atoms. Examples of such C1-12 alkyl hydroperoxides are tert-pentyl hydroperoxide (also known under the name tert-amyl hydroperoxide), 1,1,3,3-tetramethylbutyl hydroperoxide and tert-butylhydroperoxide (TBHP). Most preferably, TBHP is selected as the oxidizing agent. This oxidizing agent showed good results with respect to safety and yield, in particular when the oxidation reaction was conducted in dichloromethane.

The organic hydroperoxide is preferably contacted with the oxo-pyrimidine carbonitrile of formula (II) in the form of an aqueous solution, i.e. a solution of the organic hydroperoxide in water. For example, such a solution is added to and/or mixed with the intermediate mixture or organic extract. The aqueous solution may have a concentration of at least 25 wt. %, preferably at least 50 wt. %, even more preferably at least 60 wt. % of the organic hydroperoxide. Good results have for example been obtained using a 70% TBHP solution. The aqueous solution may be formed by dissolving the organic hydroperoxide in water. The aqueous solution can also be formed by adding an organic hydroperoxide salt to water.

The organic hydroperoxide may also be contacted with the compound of formula (II) in the form of a salt. Examples of a suitable organic hydroperoxide are peroxy acid salts, such as monoperoxyphthalates. Magnesium salts of monoperoxyphthalates are most common, such as magnesium bis(monoperoxyphthalate) hexahydrate. The organic hydroperoxide salt may be contacted with the intermediate mixture or organic extract in solid form or in dissolved form. When added in dissolved form, the organic hydroperoxide may be added as a solution in water or as an organic solution, for example dissolved in the first or second organic solvent.

Another important part of the invention lies in the selection of the organic solvents. Firstly, it is important that the oxidation step is conducted in an organic solvent, the presence of which does not have a significant negative effect on the oxidizing agent. Secondly, the compound of formula (II) should be sufficiently soluble in the organic solvent wherein the oxidation step is conducted. Preferably, the compound of formula (II) has a solubility in the organic solvent wherein the oxidation step is conducted of at least 10 g/l, more preferably at least 50 g/l at 20° C., most preferably at least 100 g/l at 20° C. Thirdly, the organic solvent is preferably immiscible with water. If the organic solvent is miscible with water, this may complicate purification and isolation of the compound of formula (III) from the reaction mixture. Suitable examples of organic solvents wherein the oxidation step may be conducted are dichloromethane, toluene and acetonitrile. In case an extraction step is conducted, the second organic solvent will be the organic solvent wherein the oxidation is conducted. Otherwise, the first organic solvent will be the organic solvent wherein the oxidation is conducted.

Furthermore, the first organic solvent should be a solvent suitable for conducting the reaction to form the compound of formula (II) in. The first organic solvent is preferably a polar solvent. The first organic solvent may be selected from the group consisting of alcohols, N-Methyl-2-pyrrolidone (NMP), dimethylsulfoxide (DSMO), sulfolane and strong polar solvents such as formic acid, acetic acid, acetonitrile and acetone. Preferably, the first organic solvent is an alcohol, which alcohol may have from 1 to 12 carbon atoms, preferably from 1 to 6 carbon atoms. Suitable examples of alcohols are methanol, ethanol, n-propanol, isopropanol and butanol (e.g. n-butanol). It is desirable to use methanol as the first solvent. The use of this solvent resulted in a good yield of oxo-pyrimidine-carbonitrile. Dichloromethane may also be selected as the first organic solvent. Although the reaction to form the compound of formula (II) will be very slow when conducted in this solvent, the choice for this solvent has the advantage that no extraction needs to be conducted. Accordingly, the first reaction and the oxidation reaction can be conducted in the same reaction vessel.

Since the requirements for the first solvent and the solvent wherein the oxidation is conducted differ from each other, the process of the invention preferably comprises an extraction step, wherein the compound of formula (II) is extracted from the first solvent into a second solvent. For this purpose, any suitable liquid-liquid extraction technique known in the art may be used.

In case no extraction is conducted, the first organic solvent may be dichloromethane, acetonitrile, methanol or tetrahydrofuran.

In case extraction is conducted, the second organic solvent should be immiscible with the first organic solvent, such that extraction of the oxo-pyrimidine-carbonitrile can be conducted from the first to the second organic solvent. The second organic solvent may be selected from the group consisting of dichloromethane, acetonitrile or toluene. Preferably, the second organic solvent is dichloromethane. It was found that the oxo-pyrimidine-carbonitrile of formula (II) could be efficiently extracted to dichloromethane and is also a suitable solvent in the oxidation step.

In a preferred embodiment, the first step is conducted in methanol (first organic solvent), after which the formed oxo-pyrimidine-carbonitrile of formula (II) is extracted to dichloromethane (second organic solvent). The specific choice for these solvents in combination with an organic hydroperoxide as the oxidizing agent results in an improved yield compared to the yield obtained when using steps 1 and 2 of WO 2008/151510.

The oxo-pyrimidine-carbonitrile of formula (II) generally has a low solubility in most organic solvents. Nevertheless, the first and optional second organic liquid are preferably selected such that the oxo-pyrimidine-carbonitrile has a reasonably good solubility in these solvents, such that the formation and oxidation reaction can be conducted with reasonable efficiency. Therefore, the oxo-pyrimidine-carbonitrile preferably has a solubility at 20° C. in the first organic solvent of 5 g/l, more preferably at least 50 g/l, most preferably at least 100 g/l at 20° C. In case of extraction, these minimum values also apply to the solubility of oxo-pyrimidine-carbonitrile in the second solvent.

Suitable reaction conditions for the first step (wherein the oxo-pyrimidine carbonitrile of formula (II) is formed) and for the oxidation step will be described below.

In the first step of the process of the invention, p-fluorobenzaldehyde, 4-methyl-3-oxopentanenitrile and urea are reacted to form the compound of formula (II). The reaction mixture in the first step comprises the three reactants described above and a first organic solvent. During the reaction, the compound of formula (II) is formed, which will typically dissolve at least partially in the first solvent. Thus, the reaction results in a mixture comprising the compound of formula (II) in dissolved form. This mixture is referred to herein as the intermediate mixture.

The molar amount 4-methyl-3-oxopentanenitrile used in the reaction may be equal to 0.5-2, preferably 0.8-1.2 times the amount of p-fluorobenzaldehyde used.

The molar amount urea used in the reaction may be equal to 1.5-2.5, preferably 1.8-2.2 times the amount of p-fluorobenzaldehyde used.

The reaction mixture in the first step of the process of the invention may comprise one or more organic solvents. Preferably, at least 50 wt. %, preferably at least 75 wt. %, more preferably at least 90 wt. %, even more preferably at least 95 wt. %, even more preferably at least 99 wt. % of the organic solvents present in the oxidation mixture is the first solvent.

The reaction mixture in the first step of the process of the invention may comprise a strong acid, such as strong organic acids or acidic resins. Suitable examples of strong organic acids are sulphuric acid, hydrochloric acid, p-toluenesulphonic acid, benzenesulphonic and methanesulphonic acid. A suitable example of an acidic resin is an acidic ion-exchange resin. Acidic resins are commercially available, e.g. under the name Amberlyst. Alternatively, the reaction mixture may comprise a weak acid, preferably a weak organic acid, which may be selected from acetic acid, benzoic acid and pivalic acid. The molar amount of acid used in the reaction may be equal to 0.05-1 times, preferably 0.1-0.5 times of the molar amount of p-fluorobenzaldehyde used.

The reaction mixture may further comprise a catalyst. The catalyst may be a metal salt. The metal may be a chloride salt, such as copper, iron or zinc chloride. Preferably, the catalyst is a metal salt selected from the group consisting of copper(I)chloride, iron(III) trichloride and zinc(II)chloride. Most preferably, copper(I)chloride is used. The catalyst may be present in a molar amount less than 0.2 times, preferably 0.001-0.1 times, the starting amount of p-fluorobenzaldehyde.

The reaction between p-fluorobenzaldehyde, 4-methyl-3-oxopentanenitrile and urea may be conducted at a temperature of 30-100° C., preferably 50-80° C., more preferably 60-70° C. The reaction of step 1 may be conducted for at least 1 hour, preferably at least 5 hours, more preferably at least 10 hours.

In addition to the first organic solvent, other organic solvents may be present. In this case, at least 50 wt. %, more preferably at least 80 wt. %, even more preferably at least 95 wt. % of the total amount of organic solvents present in the reaction mixture can be attributed to the first organic solvent.

In case no extraction is conducted, the intermediate mixture is used as the reactant in the oxidation step. In case of extraction, the compound of formula (II) is extracted from the first organic solvent to the second organic solvent prior to oxidizing. Such a step will result in a solution comprising the second organic solvent and the compound of formula (II) in dissolved form. This mixture may be referred to as the organic extract. The organic extract will then be used as the reactant in the oxidation step.

The intermediate mixture or organic extract may be concentrated or diluted prior to oxidizing. This may for example be desirable to obtain sufficiently low or high concentrations in the oxidation step. The intermediate mixture may also be concentrated or diluted in order to increase extraction efficiency.

The compound of formula (II) is oxidized by contacting the intermediate mixture or the organic extract with the organic hydroperoxide. This will result in an oxidation reaction mixture comprising the two reactants, an organic solvent (which is either the first or second solvent, dependent on whether extraction was conducted) and typically also water (in which solvent the organic hydroperoxide is typically dissolved). The oxidation results in the compound of formula (II) being converted to the compound of formula (III).

The molar amount organic hydroperoxide used in the reaction may be equal to 0.5-10, preferably 0.8-5, more preferably 1-3, even more preferably 1.2-2 times the amount of p-fluorobenzaldehyde used.

The organic hydroperoxide is preferably contacted in the form of a solution of the organic hydroperoxide in water. The solution may be dosed to the intermediate mixture over a period of time of at least 1 hour, preferably at least 2 hours.

The oxidation reaction mixture may comprise one or more organic solvents. Preferably, at least 50 wt. %, preferably at least 75 wt. %, more preferably at least 90 wt. %, even more preferably at least 95 wt. %, even more preferably at least 99 wt. % of the organic solvents present in the oxidation mixture is the first solvent (in case no extraction is conducted) or the second solvent (in case extraction is conducted).

The oxidation reaction may be conducted in the presence of a base. The base may be a sodium or potassium salt. The base may be salt of a carbonate, bicarbonate or hydroxide, for example selected from K₂CO₃, Na₂CO₃, NaHCO₃, NaOH and KOH. Preferably, an excess amount is present in the oxidation reaction mixture. It is not particularly critical how the base is provided to the oxidation reaction mixture. The base may for example be added to the intermediate mixture or organic extract prior to contacting it with the organic hydroperoxide. The base may also be added to the reaction mixture after contact said contacting.

The oxidation reaction may be conducted in the presence of a catalyst, typically a metal salt. The catalyst is preferably a copper, palladium, iron, zinc or ruthenium catalyst. The catalyst may for example be selected from CuCl₂, CuCl, Cu(OAc)₂, CuSO₄, CuO, CuNO₃, Pd(OAc)₂, Pd/C, Fe(OAc)₃, Fe(OAc)₂, FeCl₃, ZnCl₂, Zn(OAc)₂ or RuCl₃. Preferably the same catalyst is used as in the oxidation step, for example copper(I)chloride. It is not particularly critical how the base is provided to the oxidation reaction mixture. The catalyst may for example be added to the intermediate mixture or organic extract prior to contacting it with the organic hydroperoxide. The catalyst may also be added to the reaction mixture after contact said contacting. The catalyst may be present in the reaction mixture in a molar amount less than 0.2 times, preferably 0.001-0.1 times, of the starting amount of the compound of formula (II).

The oxidation reaction may be conducted at a temperature of 10-60° C., preferably 25-45° C. The reaction may be conducted for at least 15 minutes, preferably at least 30 minutes, more preferably at least 1 hour.

The oxidation may be ended by quenching the oxidation reaction mixture with an aqueous solution comprising a base, such as Na₂SO₃ or aqueous NH₄Cl (75 mL). If required, the pH of the reaction mixture may be adjusted to a value between 7 and 8, e.g. about 7.5.

After oxidation or quenching, the oxidation reaction mixture comprising the compound of formula (III) may be further processed by concentrating the organic phase. Further, one or more washing steps may be conducted, e.g. using water, dichloromethane and/or toluene. The compound of formula (III) may be isolated by filtration or crystallization (e.g. from a toluene solution).

In case the oxidation reaction mixture comprises water and an organic solvent, the water and organic solvent may be separated from each other by salt-induced phase separation. This may in particular be desirable when the organic solvent is miscible with water, such as in case of acetonitrile and tetrahydrofuran. Salt-induced phase separation may be conducted by adding an inorganic salt (e.g. (NaCl)) to the oxidation reaction mixture. As a result of the salt addition, the aqueous and organic phase will separate. After phase-separation, the water phase comprising the salt and the organic phase comprising the compound of formula (III) can easily be separated from each other, e.g. by decantation. Salt-induced phase separation is preferably applied in the process of the invention in the specific case when no extraction step is conducted, the first organic solvent is miscible with water, and the organic hydroperoxide is added as an aqueous solution. Once the compound of formula (III) is isolated, it may be reacted further to form the compound of formula (I).

The compound of formula (III) may first be converted to the compound of formula (IV).

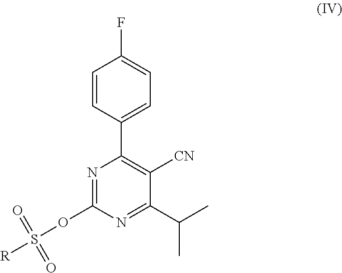

This may for example be achieved by contacting a mixture of the compound of formula (III) and an organic solvent (preferably toluene) with a sulfonic acid derivative such as an organic sulfonyl halide. The compound of formula (IV) may then be converted to the compound of formula (V) by contacting the compound of formula (IV) with N-methylmethane sulfonamide.

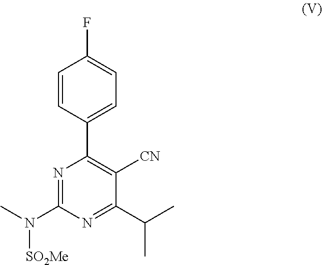 [SMILES: CC(C)c1nc(N(C)S(C)(=O)=O)nc(-c2ccc(F)cc2)c1C#N]

Such steps are known in the art, for example from WO 2008/151510. Preferably both steps are conducted in toluene.

The compound of formula (V) may subsequently be subjected to a reduction step in order to form the compound of formula (I). Any reducing agent suitable for the conversion to the compound of formula (V) may be used, for example those described in WO 2008/151510. Preferably however, diisobutylaluminium hydride (DIBALH) is used as the reducing agent using toluene as the solvent.

The compound of formula (I) may subsequently be subjected to a Julia-Kocienski type olefination leading to rosuvastatin as described e.g. in WO 2013/083718. More specifically, in this approach the compound of formula (I) is reacted with a compound of general formula (VI) or of general formula (VII)

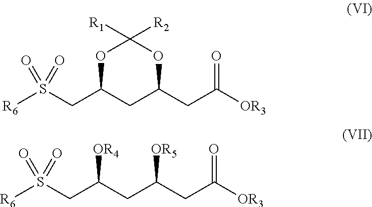

to give a compound of general formula (VIII) or of general formula (IX) respectively.

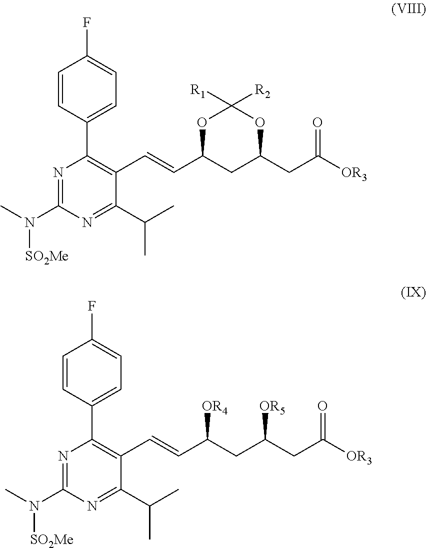

The latter compounds may be subsequently converted to rosuvastatin calcium of formula (X) by deprotection, for example using acid, followed by treatment with a compound comprising calcium ions, such as calcium acetate or calcium chloride or the like.

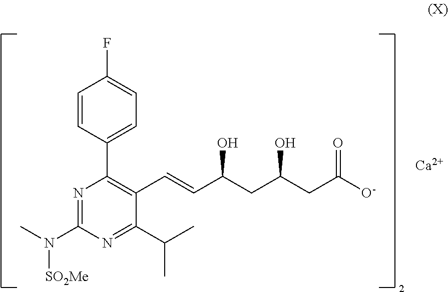

In the above conversions R₁ to R₅ each independently stand for an alkyl with for instance 1 to 12 carbon atoms, preferably 1 to 6 carbon atoms, an alkenyl with for instance 1 to 12 carbon atoms, preferably 1 to 6 carbon atoms, a cycloalkyl with for instance 3 to 7 carbon atoms, a cycloalkenyl with for instance 3 to 7 carbon atoms, an aryl with for instance 6 to 10 carbon atoms or an aralkyl with for instance 7 to 12 carbon atoms, each of R₁ to R₅ may be substituted. R₁ and R₂ may form a ring together with the carbon atom to which they are bound. R₆ is an aryl group that for instance is suitable for a one-pot or modified Julia-Kocienski olefination. Suitable aryl groups are e.g. described in P. R. Blakemore, J. Chem. Soc., Perkin Trans. 1, 2002, 2563. Preferred aryl groups include tetrazole, substituted phenyl and benzimidazole type compounds. Specific examples of preferred aryl groups include, pyridine-2-yl, pyrimidin-2-yl, benzo-thiazol-2-yl, 1-methyl-1H-tetrazol-5-yl, 1-phenyl-1H-tetrazol-5-yl, 1-tert-butyl-1-H-tetrazol-5-yl, 3,5-bis(trifluoromethyl)phenyl-1-yl, 1-methylimidazol-2-yl, benzimidazol-2-yl, 4-methyl-1,2,4-triazol-3-yl and iso-quinolin-1-yl. Most preferred aryl groups are 1-methyl-1H-tetrazol-5-yl, 1-phenyl-1H-tetrazol-5-yl, 1-tert-butyl-1-H-tetrazol-5-yl, benzo-thiazol-2-yl, and 3,5-bis(trifluoromethyl)phenyl-1-yl.

The invention is further illustrated by the following examples.

### EXAMPLES

### Example 1

### Oxo-Pyrimidine-Carbonitrile Formation

This example shows the preparation of 4-(4-fluorophenyl)-6-isopropyl-2-oxo-1,2,3,4-tetrahydropyrimidine-5-carbonitrile from p-fluorobenzaldehyde, 4-methyl-3-oxopentanenitrile and urea conducted in methanol. The example corresponds to step 1 of WO 2008/151510. The reaction mechanism is as follows.

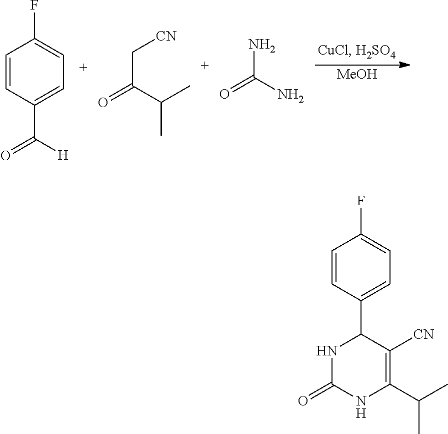

To a reactor of 2 L was added p-fluorobenzaldehyde (156 g, 1.26 mol), 4-methyl-3-oxopentanenitrile (140 g, 1.26 mol) and MeOH (330 mL). To the clear mixture was added urea (151 g, 2.52 mol) and Cu(I)Cl (1.25 g, 12.6 mmol), followed by addition of concentrated H₂SO₄ in 5 min (10.07 mL, 0.19 mol). The reaction mixture was heated in 30 min to 65° C. The now clear brownish solution was stirred and kept at 65° C. for 64 h. The reaction mixture was cooled to 20° C. in 3 h and stirred for 2 h at this temperature.

The precipitated solid was filtered and washed with MeOH (2×60 mL). The crude solid was suspended in 135 mL of MeOH and 470 mL of water. The slurry is stirred and heated at 65° C. for 1 h and then cooled in 2 h to 20° C. The solid is isolated by filtration, washed with 3 portions of a mixture of 22.5 mL MeOH/90 mL of water. After drying, 4-(4-fluorophenyl)-6-isopropyl-2-oxo-1,2,3,4-tetrahydropyrimidine-5-carbonitrile was obtained as a white solid (191 g, yield 58% based on 4-methyl-3-oxopentanenitrile). ¹H NMR (300 MHz, CDCl₃) δ 1.20-1.26 (d, 6H, J=6.9 Hz), 3.06-3.11 (m, 1H), 5.15 (s, 1H), 5.74 (bs, 1H), 7.07-7.13 (m, 2H), 7.27-7.35 (m, 2H), 8.35 (bs, 1H). Although it appears from the Examples of WO 2008/151510 that a yield of up to 83% can be obtained, such a high yield could not be obtained when trying to rework WO 2008/151510. The relative low yield obtained in the above Example (58%) can be attributed to the difficulty in isolating the oxo-pyrimidine-carbonitrile crystals from the reaction mixture.

### Example 2

### Oxo-Pyrimidine-Carbonitrile Oxidation with HNO₃

This example shows the preparation of 4-(4-fluorophenyl)-2-hydroxy-6-isopropylpyrimidine-5-carbonitrile from 4-(4-fluorophenyl)-6-isopropyl-2-oxo-1,2,3,4-tetrahydropyrimidine-5-carbonitrile using HNO₃ as an oxidizing agent. The example corresponds to step 2 of WO 2008/151510. The reaction mechanism is as follows.

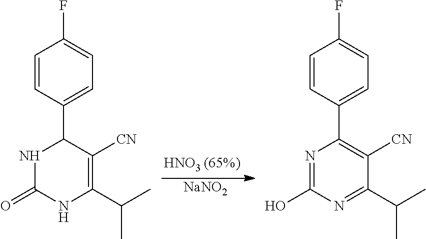

To a reactor is added an aqueous solution of 65% conc. HNO₃ (129.2 g, 1.38 mol). The reaction mixture is cooled to 10° C. and NaNO₂ (0.92 g, 0.013 mol) was added. Then 4-(4-fluorophenyl)-6-isopropyl-2-oxo-1,2,3,4-tetrahydropyrimidine-5-carbonitrile (40 g, 0.15 mol) is dosed in 80 min keeping the temperature below 10° C. The reaction is stirred for 3 h at 10° C. Then water (360 mL) is added and the pH adjusted to 5 with 50% aqueous NaOH keeping the temperature below 15° C. The precipitated solid was isolated by filtration and washed with water (2×50 mL). After drying, 4-(4-fluorophenyl)-2-hydroxy-6-isopropylpyrimidine-5-carbonitrile is obtained as a slightly yellow solid (36.0 g, yield 90%). ¹H NMR (300 MHz, CDCl₃) δ 1.42-1.44 (d, 6H, J=7.0 Hz), 3.40-3.43 (m, 1H), 7.14-7.20 (m, 2H), 7.90-7.95 (m, 2H), 8.50-9.50 (bs, 1H).

### Example 3

### Oxo-Pyrimidine-Carbonitrile Oxidation with TBHP

This Example shows the preparation of 4-(4-fluorophenyl)-2-hydroxy-6-isopropylpyrimidine-5-carbonitrile from 4-(4-fluorophenyl)-6-isopropyl-2-oxo-1,2,3,4-tetrahydropyrimidine-5-carbonitrile using tert-butylhydroperoxide (TBHP) as an oxidizing agent. The reaction mechanism is as follows.

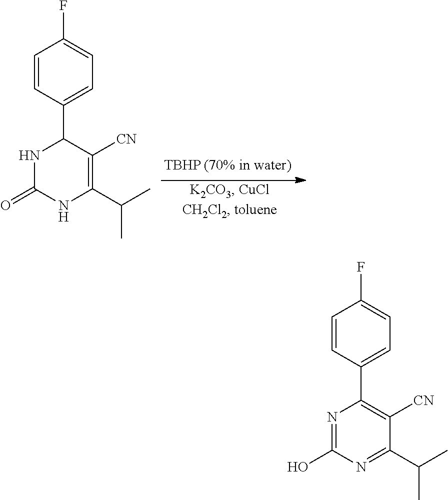 [SMILES: CC(C)C1=C(C#N)C(c2ccc(F)cc2)NC(=O)N1 ; "K2CO3, CuClCH2Cl2, toluene" ; CC(C)c1nc(O)nc(-c2ccc(F)cc2)c1C#N]

To a reactor is added CH₂Cl₂ (150 mL), 4-(4-fluorophenyl)-6-isopropyl-2-oxo-1,2,3,4-tetrahydropyrimidine-5-carbonitrile (16.2 g, 62.5 mmol), K₂CO₃ (0.6 g, 4.4 mol) and CuCl (0.06 g, 0.6 mmol). The reaction mixture is heated to 35° C. under stirring, whereupon a clear solution is obtained. Next, TBHP (14.6 mL tert-butylhydroperoxide, 70% in water, 106.2 mmol) is dosed during 3 h. When dosing is completed, the reaction mixture is stirred for 1 h at 35° C., cooled and stirred for 16 h at 20° C., whereupon the color of the reaction mixture has changed from clear yellow to turbid greenish. Next the reaction mixture is quenched by addition of 0.5 M aqueous Na₂SO₃ (150 mL) and 25 w/w % of aqueous NH₄Cl (75 mL). If required, the pH is adjusted to 7.5. The layers are separated and the water layer is washed with CH₂Cl₂ (2×50 mL). The combined organic layers are concentrated under vacuum. To the residue is added toluene (100 mL) and concentrated to about 50 mL. Then toluene (100 mL) is added and the slurry stirred for 1 h at 20° C. The precipitated solid is isolated by filtration and washed with toluene (3×5 mL). After drying, 4-(4-fluorophenyl)-2-hydroxy-6-isopropylpyrimidine-5-carbonitrile is obtained as a white/light green solid (14.8 g, yield 92%).

It can be concluded from Examples 2 and 3 that the oxidation of the oxo-pyrimidine-carbonitrile can be conducted at least as efficient with TBHP as with HNO₃.

### Example 4

### Integrated Oxo-Pyrimidine-Carbonitrile Formation and Oxidation

This Example shows the proof of concept for the preparation of 4-(4-fluorophenyl)-2-hydroxy-6-isopropylpyrimidine-5-carbonitrile from p-fluorobenzaldehyde, 4-methyl-3-oxopentanenitrile and urea, wherein the oxo-pyrimidine-carbonitrile formation and oxidation with TBHP have been integrated. The oxo-pyrimidine-carbonitrile was not isolated after formation, but is to be directly oxidized with TBHP. The reaction mechanism is as follows.

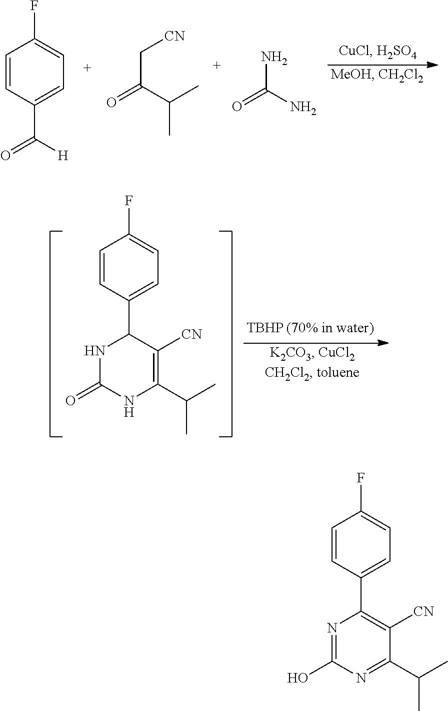

To a reactor was added p-fluorobenzaldehyde (16.8 g, 135 mmol), 4-methyl-3-oxopentanenitrile (15.0 g, 135 mmol), MeOH (35 mL), urea (16.2 g, 270 mmol) and Cu(I)Cl (0.13 g, 1.3 mmol), followed by addition of concentrated H₂SO₄ (1.12 mL, 20.2 mmol) in 5 min. The reaction mixture was heated to 62° C. and stirred at this temperature for 85 h. The reaction mixture is cooled to 35° C., CH₂Cl₂ (60 mL) and water (60 mL) were added to wash off any excess Cu(I)Cl. After stirring for 30 min, the phases are separated. The water layer is washed with CH₂Cl₂ (50 mL). The combined organic layers are used in the next oxidation step. Total 143 g containing 22.7 g 4-(4-fluorophenyl)-6-isopropyl-2-oxo-1,2,3,4-tetrahydropyrimidine-5-carbonitrile (65% yield, 15.8 w/w %).

The carbonitrile solution in the organic layers can be used in an oxidation step with TBHP, such as described above in Example 3. In view of the increased yield of the oxo-pyrimidine-carbonitrile in the organic fraction (65%) compared to the yield obtained in Example 1 (58%), it is to be expected that this will result in an increased overall yield for steps 1 and 2 which was confirmed in the below Example 5.

### Example 5

### Preparation of 4-(4-fluorophenyl)-2-hydroxy-6-isopropylpyrimidine-5-carbonitrile without isolation of 4-(4-fluorophenyl)-6-isopropyl-2-oxo-1,2,3,4-tetrahydropyrimidine-5-carbonitrile (oxidation with TBHP)

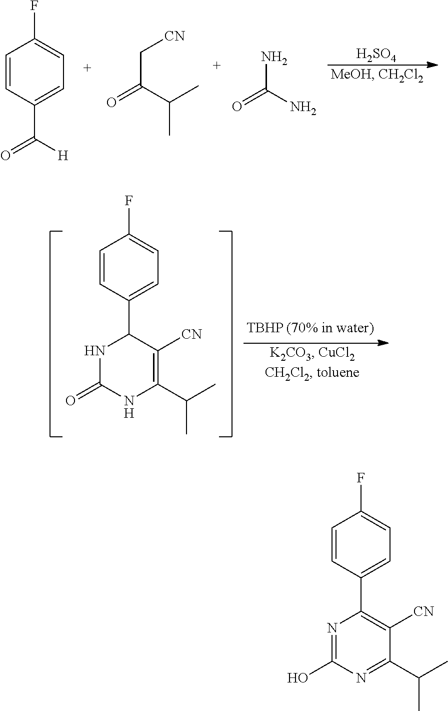

To a reactor was added p-fluorobenzaldehyde (8.4 g, 68 mmol), 4-methyl-3-oxopentanenitrile (7.5 g, 67 mmol), MeOH (18 mL), urea (8.1 g, 135 mmol) followed by concentrated H₂SO₄ (1.1 mL). The reaction mixture was heated to 70° C. and stirred at this temperature for 35 h. The reaction mixture was cooled to 35° C., CH₂Cl₂ (60 mL) and water (60 mL) were added. After stirring for 30 min, the phases were separated. The water layer was washed with CH₂Cl₂ (40 mL). The combined organic layers were used in the next oxidation step. Total 124 g solution containing 14.5 g 4-(4-fluorophenyl)-6-isopropyl-2-oxo-1,2,3,4-tetrahydropyrimidine-5-carbonitrile (82% yield) was obtained. The solution was heated to 35° C. and K₂CO₃ (0.49 g) and CuCl₂ (0.05 g) were added. Next 11.8 mL of TBHP as a 70% solution in water) was dosed in 3 h. When dosing was completed, the reaction mixture was stirred for 1 h at 35° C., cooled and stirred for 16 h at 20° C. The reaction mixture was quenched by addition of 0.5 M aqueous Na₂SO₃ (100 mL) and 25 w/w % of aqueous NH₄Cl (50 mL). The organic layer was separated. The aqueous layer was extracted with CH₂Cl₂ (50 mL). The combined organic layers were concentrated until 50 mL. Then toluene (80 mL) was added, followed by concentration under vacuum to remove the CH₂Cl₂. The resulting slurry was stirred for 1 h at 20° C. The precipitated solid was isolated by filtration, washed with toluene (2×10 mL). After drying, 4-(4-fluorophenyl)-2-hydroxy-6-isopropylpyrimidine-5-carbonitrile was obtained as a white solid (11.7 g, yield 68% based on 4-methyl-3-oxopentanenitrile).

1. Process for preparing a compound of formula (III), comprising the steps of:
(a) reacting p-fluorobenzaldehyde, 4-methyl-3-oxopentanenitrile and urea in a first reaction mixture comprising a first organic solvent, thereby obtaining an intermediate mixture comprising the compound of formula (II) dissolved in the first organic solvent; and 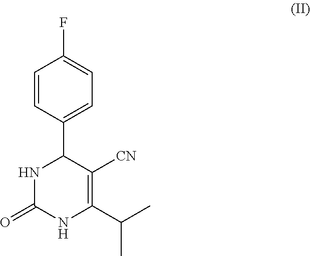 [SMILES: CC(C)C1=C(C#N)C(c2ccc(F)cc2)NC(=O)N1]
(b) oxidizing the compound of formula (II) with an organic hydroperoxide, thereby obtaining the compound of formula (III), 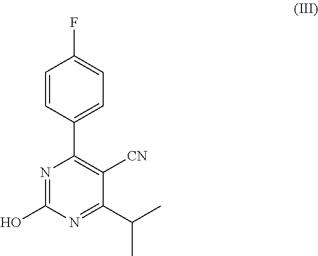 [SMILES: CC(C)c1nc(O)nc(-c2ccc(F)cc2)c1C#N]
wherein the compound of formula (II) is kept in dissolved form between its formation and oxidation.

2. Process according to claim 1 wherein said first organic solvent is selected from the group consisting of alcohols, N-methyl-2-pyrrolidone, dimethylsulfoxide, sulfolane and polar solvents.

3. Process according to claim 1 wherein step (a) is conducted at a temperature of from 50-80° C.

4. Process according to claim 1 further comprising extracting said compound of formula (II) from said intermediate mixture to a second organic solvent, thereby forming an organic extract comprising the compound of formula (II) dissolved in said second organic solvent; and oxidizing said compound of formula (II) by contacting said organic extract with an organic hydroperoxide, thereby obtaining said compound of formula (III).

5. Process according to claim 4 wherein said second organic solvent is selected from the group consisting of dichloromethane, toluene and acetonitrile.

6. Process according to claim 1 wherein said organic hydroperoxide is tert-butylhydroperoxide.

7. Process according to claim 1, wherein said organic hydroperoxide is contacted in the form of an aqueous solution with said intermediate mixture or organic extract.

8. Process according to claim 1 wherein said oxidation reaction is conducted in the presence of excess base and/or a catalyst.

9. Process according to claim 8 wherein said base is selected from the group consisting of K₂CO₃, Na₂CO₃, NaHCO₃, NaOH and KOH and said catalyst is selected from the group consisting of copper, palladium, iron, zinc and ruthenium catalysts.

10. Process according to claim 1 wherein said first reaction mixture comprises an acid selected from the group consisting of acetic acid, benzoic acid, pivalic acid, sulphuric acid, hydrochloric acid, p-toluenesulphonic acid, benzenesulphonic, methanesulphonic acid and acidic resins.

11. Process according to claim 1, wherein said intermediate mixture is concentrated or diluted prior to oxidizing.

12. Process according to claim 1 wherein said compound of formula (III) is converted to a compound of formula (V) 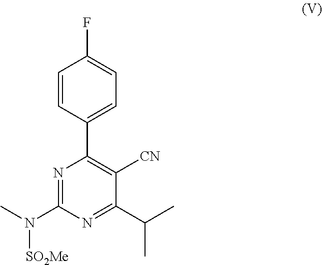 [SMILES: CC(C)c1nc(N(C)S(C)(=O)=O)nc(-c2ccc(F)cc2)c1C#N]
by reacting with a sulfonic acid derivative followed by reaction with N-methylmethane sulfonamide.

13. Process according to claim 12 further comprising treating said compound of formula (V) with a reducing agent to obtain a compound of formula (I).

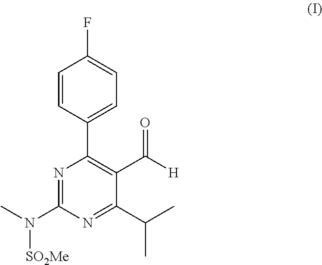 [SMILES: CC(C)c1nc(N(C)S(C)(=O)=O)nc(-c2ccc(F)cc2)c1C=O]

14. Process according to claim 13 further comprising treating said compound of formula (I) with a compound of formula (VI) or a compound of formula (VII) 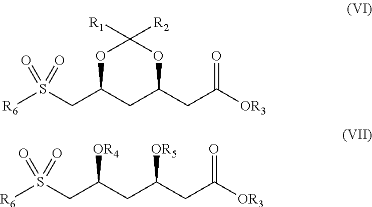
to obtain a compound of formula (VIII) or a compound of formula (IX), respectively.

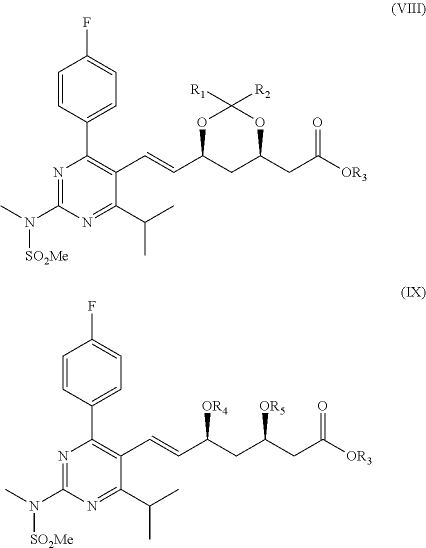

15. Process according to claim 14 further comprising subjecting said compound of formula (VIII) or said compound of formula (IX) to deprotection to obtain rosuvastatin calcium of formula (X). [chemical structure]
